(19) United States
(12) Patent Application Publication
(10) Pub. No.: US 2025/0146964 A1
(43) Pub. Date: May 8, 2025

(54) THERMAL IMAGING METHOD FOR CRACK AND HOLE DETECTION IN SEMICONDUCTOR DEVICES

(71) Applicant: Orbotech Ltd., Yavne (IL)

(72) Inventors: Elkana Porat, Yavne (IL); Ronen Yogev, Hulda (IL); Nir Turko, Yavne (IL); Yuri Paskover, Binyamina (IL); Kristiaan Van Rossen, Yavne (IL); Bert Vangilbergen, Yavne (IL); Federico Buja, Yavne (IL); Kjell Cnops, Yavne (IL)

(21) Appl. No.: 18/618,417

(22) Filed: Mar. 27, 2024

Related U.S. Application Data
(60) Provisional application No. 63/547,147, filed on Nov. 3, 2023.

(51) Int. Cl. G01N 25/72; G01J 5/00; G01J 5/48; G01N 21/88; G01N 21/95

(52) U.S. Cl. CPC G01N 25/72; G01J 5/48; G01N 21/8806; G01N 21/8851; G01N 21/9505; G01J 2005/0077

(57) ABSTRACT

The system includes a first laser light source configured to emit laser light, a first focusing lens configured to direct the laser light from the first laser light onto a first face of a workpiece, a thermal camera configured to capture a thermal image of a second face of the workpiece that is orthogonal to the first face, and a processor configured to identify a crack in the workpiece based on the thermal image.

### CROSS-REFERENCE TO RELATED APPLICATIONS

This application claims priority to the provisional patent application filed Nov. 3, 2023, and assigned U.S. App. No. 63/547,147, the disclosure of which is hereby incorporated by reference.

### FIELD OF THE DISCLOSURE

This disclosure relates to semiconductor manufacturing and, more particularly, to inspection processes for detection of defects in semiconductor and solid state battery manufacturing.

### BACKGROUND OF THE DISCLOSURE

Evolution of the electronics manufacturing industry is placing greater demands on yield management and, in particular, on metrology and inspection systems. Critical dimensions continue to shrink, yet the industry needs to decrease time for achieving high-yield, high-value production. Minimizing the total time from detecting a yield problem to fixing it maximizes the return-on-investment for an electronics manufacturer.

Fabricating semiconductor devices, such as logic and memory devices, typically includes processing a semiconductor wafer using a large number of fabrication processes to form various features and multiple levels of the semiconductor devices. For example, lithography is a semiconductor fabrication process that involves transferring a pattern from a reticle to a photoresist arranged on a semiconductor wafer. Additional examples of semiconductor fabrication processes include, but are not limited to, chemical-mechanical polishing (CMP), etching, deposition, and ion implantation. An arrangement of multiple semiconductor devices fabricated on a single semiconductor wafer may be separated into individual semiconductor devices.

Inspection processes are used at various steps during electronics manufacturing to detect defects on wafers, electronic devices, or electrical circuits to promote higher yield in the manufacturing process and, thus, higher profits. Inspection has always been an important part of fabricating electronic devices such as integrated circuits (ICs) and printed circuit boards (PCBs), including assembled PCBs. However, as emerging markets (e.g., electric vehicles and others) push for increased reliability requirements, detection of structural defects of packaged semiconductor device of decreasing size has become necessary, because even relatively small defects may cause unwanted failure of the devices during its complete life-cycle.

During a dicing process of separating individual semiconductor devices from a single wafer, cracks can form along the edges around the perimeter of the semiconductor device. To identify these cracks, a single side wall of the device is illuminated with near infra-red (NIR) or short wave infra-red (SWIR), and a camera captures an image on the opposite side wall. The NIR or SWIR light is transmitted through a translucent semiconductor device, and the cracks cast shadows that can be detected in the image captured by the camera on the opposite side. However, this detection method is limited to translucent semiconductor devices and does not work with opaque semiconductor devices.

Therefore, what is needed is a method capable of detecting defects in opaque semiconductor devices.

### BRIEF SUMMARY OF THE DISCLOSURE

An embodiment of the present disclosure provides a system comprising: a first laser light source configured to emit laser light; a first focusing lens configured to direct the laser light from the first laser light source onto a first face of a workpiece; a thermal camera configured to capture a thermal image of a second face of the workpiece, wherein the second face is orthogonal to the first face; and a processor configured to identify a crack in the workpiece based on the thermal image.

In some embodiments, the system further comprises a first scanning mirror configured to direct the laser light from the first laser light source to scan across the first face of the workpiece.

In some embodiments, the system further comprises a stage. The workpiece may be disposed on the stage, and the stage may be movable to scan the laser light from the first laser light source across the first face of the workpiece.

In some embodiments, the first face has a smaller cross-sectional area than the second face. The laser light may have a diameter that is less than or equal to a thickness of the first face of the workpiece.

In some embodiments, the first face has a larger cross-sectional area than the second face. The first focusing lens may be configured to direct the laser light onto the first face of the workpiece near an edge adjoining the first face and the second face.

In some embodiments, the system further comprises a second laser light source configured to emit laser light and a second focusing lens configured to direct the laser light from the second laser light source onto the first face of the workpiece at a position that is offset from the laser light from the first laser light source.

In some embodiments, the system further comprises a third laser light source configured to emit laser light and a third focusing lens configured to direct the laser light from the third laser light source onto a third face of the workpiece. The third face may be orthogonal to the second face and parallel to the first face.

In some embodiments, the system further comprises a fourth laser light source configured to emit laser light and a fourth focusing lens configured to direct the laser light from the fourth laser light source onto a fourth face of the workpiece. The fourth face may be orthogonal to the second face and the first face.

In some embodiments, the system further comprises a fifth laser light source configured to emit laser light and a fifth focusing lens configured to direct the laser light from the fifth laser light source onto a fifth face of the workpiece. The fifth face may be orthogonal to the second face and parallel to the fourth face.

In some embodiments, the thermal camera is a forward looking infrared (FLIR) camera.

In some embodiments, the processor is configured to identify the crack in the workpiece based on the thermal image by: obtaining an intensity of each pixel of the thermal image along an edge of the workpiece adjoining the first face and the second face; deriving a gradient map representing a spatial derivative of a temperature profile along the edge of the workpiece adjoining the first face and the second face using the intensity of each pixel of the thermal image; and determining that the crack in the workpiece exists where there is a peak in the gradient map.

Another embodiment of the present disclosure provides a method comprising: emitting laser light from a first laser light source; directing the laser light from the first laser light source onto a first face of a workpiece with a first focusing lens; capturing a thermal image of a second face of the workpiece with a thermal camera, wherein the second face is orthogonal to the first face; and identifying, with a processor, a crack in the workpiece based on the thermal image.

In some embodiments, the method may further comprise scanning the laser light from the first laser light source across the first face of the workpiece with a first scanning mirror.

In some embodiments, the method may further comprise moving a stage relative to the laser light to scan the laser light from the first laser light source across the first face of the workpiece. The workpiece may be disposed on the stage.

In some embodiments, the method may further comprise rotating the stage about an axis perpendicular to the laser light from the first laser light source, such that the laser light from the first laser light source is directed onto a fourth face of the workpiece with the first focusing lens. The fourth face may be orthogonal to the second face and the first face.

In some embodiments, the method may further comprise emitting laser light from a second laser light source; and directing the laser light from the second laser light source onto the first face of the workpiece with a second focusing lens at a position that is offset from the laser light from the first laser light source.

In some embodiments, the method may further comprise emitting laser light from a third laser light source; and directing the laser light from the third laser light source onto a third face of the workpiece with a third focusing lens. The third face may be orthogonal to the second face and parallel to the first face.

In some embodiments, the method may further comprise emitting laser light from a fourth laser light source; and directing the laser light from the fourth laser light source onto a fourth face of the workpiece with a fourth focusing lens. The fourth face may be orthogonal to the second face and the first face.

In some embodiments, the method may further comprise emitting laser light from a fifth laser light source; and directing the laser light from the fifth laser light source onto a fifth face of the workpiece with a fifth focusing lens. The fifth face may be orthogonal to the second face and parallel to the fourth face.

In some embodiments, identifying, with the processor, the crack in the workpiece based on the thermal image comprises: obtaining an intensity of each pixel of the thermal image along an edge of the workpiece adjoining the first face and the second face; deriving a gradient map representing a spatial derivative of a temperature profile along the edge of the workpiece adjoining the first face and the second face using the intensity of each pixel of the thermal image; and determining that the crack in the workpiece exists where there is a peak in the gradient map.

### DESCRIPTION OF THE DRAWINGS

For a fuller understanding of the nature and objects of the disclosure, reference should be made to the following detailed description taken in conjunction with the accompanying drawings, in which:

FIG. 1 is a side view of a system according to an embodiment of the present disclosure;

FIG. 2A is a top perspective view of an exemplary workpiece of an embodiment of the present disclosure;

FIB. 2B is a bottom perspective view of the exemplary workpiece of FIG. 2A;

FIG. 3 is a top view of a system according to an embodiment of the present disclosure;

FIG. 4A is a side view of a system according to another embodiment of the present disclosure;

FIG. 4B is a top view of the system of FIG. 4A;

FIG. 5 is a top view of a system according to an embodiment of the present disclosure;

FIG. 6 is a top view of a system according to another embodiment of the present disclosure;

FIG. 7 is a flowchart of a method according to an embodiment of the present disclosure;

FIG. 8 is a flowchart of a method according to another embodiment of the present disclosure;

FIG. 9 is a flowchart of a method according to another embodiment of the present disclosure;

FIG. 10 is a flowchart of a method according to another embodiment of the present disclosure; and

FIG. 11 is a flowchart of a method according to another embodiment of the present disclosure.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

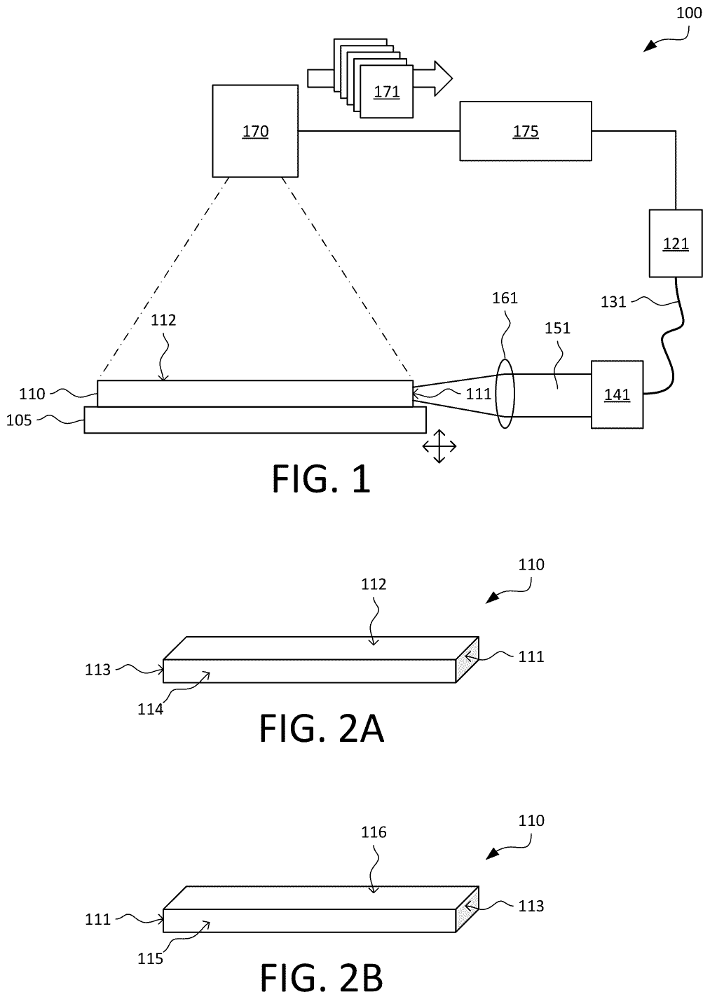

An embodiment of the present disclosure provides a system 100, as shown in FIG. 1. The system 100 may be an inspection system or a metrology system for processing or sorting a workpiece 110. The workpiece 110 may be a semiconductor wafer, die, substrate, IC, PCB, flat panel display (FPD), or one or more layers of a solid state battery, or other types of devices. The workpiece 110 may be opaque or may include one or more opaque layers. Alternatively, the workpiece 110 may be optically transparent, but may at least partially absorb laser light. As referenced herein, the workpiece 110 may be a six-sided structure comprising a first face 111, a second face 112, a third face 113, a fourth face 114, a fifth face 115, and a sixth face 116, as shown in FIG. 2A and FIG. 2B. Alternatively, the workpiece 110 may be a cylindrical structure, in which the first face 111 is a continuous annular face. The workpiece 110 may be disposed on a stage 105. For example, the sixth face 116 may be disposed on the stage 105. The stage 105 may be configured to translate (i.e., move in the X, Y, and/or Z directions) and/or rotate (e.g., along the Z axis) to change the orientation of the workpiece 110 relative to other elements of the system 100.

The system 100 may comprise a first laser light source 121. The first laser light source 121 may be a laser diode or other laser light source configured to emit laser light. The laser light may be a pulsed laser or a continuous laser beam. The first laser light source 121 may be coupled to a first optical fiber 131. The first optical fiber 131 may guide the laser light emitted by the first laser light source 121 to a first collimator 141 to produce a first laser beam 151. The first laser light source 121 may be configured to produce a first laser beam 151 having a wavelength that can be efficiently absorbed by the workpiece 110 and may have sufficient power to heat the material of the workpiece 110 by one or more ° C. For example, the first laser beam 151 may have a wavelength in the visible or near-IR spectrum and have a power of a few Watts. In an instance, while a workpiece 110 of a silicon wafer may be transparent to SWIR imaging at wavelengths of about 1100 nm to about 1300 nm, the first laser light source 121 may be configured to produce a first laser beam 151 having a wavelength of about 400 nm to 950 nm to be absorbed by the workpiece 110 and heat the workpiece 110.

The system 100 may further comprise a first focusing lens 161. In some embodiments, the first focusing lens 161 may be an f-theta lens or other type of lens. The first focusing lens 161 may be configured to direct the first laser beam 151 onto the first face 111 of the workpiece 110. For example, the first laser beam 151 may be directed along an edge of the workpiece 110 adjoining the first face 111 and the second face 112. The edge of the workpiece 110 may include the edge adjoining the first face 111 and the second face 112 itself and/or a perimeter area of along the edge, which may be, for example, less than 1 mm wide. The first focusing lens 161 may direct a laser spot from the first laser beam 151 onto the first face 111 of the workpiece 110. In some embodiments, the diameter of the laser spot may be less than or equal to a thickness of the first face 111. For example, the diameter of the laser spot may be 24 μm to 750 μm. By directing the first laser beam 151 onto the first face 111 of the workpiece 110, heat may be generated in a non-uniform temperature profile from the laser spot into the workpiece 110. For example, the temperature profile may indicate a change in temperature in the workpiece 110 of one or more ° C., such as 10° C. or more or 100° C., based on the first laser beam 151.

The system 100 may further comprise a thermal camera 170. The thermal camera 170 may be a forward looking infrared (FLIR) camera or other type of camera. The thermal camera 170 may be sensitive to the thermal radiation emitted by the workpiece. For example, the thermal camera 170 may have a wavelength sensitivity range covering part of the MWIR-LWIR range, which can be 3 μm to 15 μm. The thermal camera 170 may be directed toward the second face 112 of the workpiece 110. For example, the field of view of the thermal camera 170 may encompass the second face 112 of the workpiece 110 or a portion of the second face of the workpiece 110. The second face 112 may be orthogonal to the first face 111 of the workpiece 110. In other words, the first face 111 and the second face 112 may be adjacent faces of the workpiece 110 joined at an edge. The thermal camera 170 may be configured to capture a thermal image 171 of the second face 112 of the workpiece 110. In particular, the heat generated by the laser spot from the first laser beam 151 on the first face 111 may have a non-uniform temperature profile that extends into the workpiece 110. Thus, the thermal image 171 may reflect the non-uniform temperature profile on the second face 112 of the workpiece 110 (e.g., with the highest temperature at the position of the laser spot on the edge of the second face 112 and the temperature being reduced farther away from and along the edge adjoining the first face 111).

The system 100 may further comprise a processor 175. The processor 175 may include a microprocessor, a microcontroller, or other devices.

The processor 175 may be coupled to the components of the system 100 in any suitable manner (e.g., via one or more transmission media, which may include wired and/or wireless transmission media) such that the processor 175 can receive output. The processor 175 may be configured to perform a number of functions using the output. An inspection tool can receive instructions or other information from the processor 175. The processor 175 optionally may be in electronic communication with another inspection tool, a metrology tool, a repair tool, or a review tool (not illustrated) to receive additional information or send instructions.

The processor 175 may be part of various systems, including a personal computer system, image computer, mainframe computer system, workstation, network appliance, internet appliance, or other device. The subsystem(s) or system(s) may also include any suitable processor known in the art, such as a parallel processor. In addition, the subsystem(s) or system(s) may include a platform with high-speed processing and software, either as a standalone or a networked tool.

The processor 175 may be disposed in or otherwise part of the system 100 or another device. In an example, the processor 175 and may be part of a standalone control unit or in a centralized quality control unit. Multiple processors 175 may be used, defining multiple subsystems of the system 100.

The processor 175 may be implemented in practice by any combination of hardware, software, and firmware. Also, its functions as described herein may be performed by one unit, or divided up among different components, each of which may be implemented in turn by any combination of hardware, software and firmware. Program code or instructions for the processor 175 to implement various methods and functions may be stored in readable storage media, such as a memory.

If the system 100 includes more than one subsystem, then the different processors 175 may be coupled to each other such that images, data, information, instructions, etc. can be sent between the subsystems. For example, one subsystem may be coupled to additional subsystem(s) by any suitable transmission media, which may include any suitable wired and/or wireless transmission media known in the art. Two or more of such subsystems may also be effectively coupled by a shared computer-readable storage medium (not shown).

The processor 175 may be configured to perform a number of functions using the output of the system 100 or other output. For instance, the processor 175 may be configured to send the output to an electronic data storage unit or another storage medium. The processor 175 may be further configured as described herein.

The processor 175 may be configured according to any of the embodiments described herein. The processor 175 also may be configured to perform other functions or additional steps using the output of the system 100 or using images or data from other sources.

The processor 175 may be communicatively coupled to any of the various components or sub-systems of system 100 in any manner known in the art. Moreover, the processor 175 may be configured to receive and/or acquire data or information from other systems (e.g., inspection results from an inspection system such as a review tool, a remote database including design data and the like) by a transmission medium that may include wired and/or wireless portions. In this manner, the transmission medium may serve as a data link between the processor 175 and other subsystems of the system 100 or systems external to system 100. Various steps, functions, and/or operations of system 100 and the methods disclosed herein are carried out by one or more of the following: electronic circuits, logic gates, multiplexers, programmable logic devices, ASICs, analog or digital controls/switches, microcontrollers, or computing systems. Program instructions implementing methods such as those described herein may be transmitted over or stored on carrier medium. The carrier medium may include a storage medium such as a read-only memory, a random-access memory, a magnetic or optical disk, a non-volatile memory, a solid-state memory, a magnetic tape, and the like. A carrier medium may include a transmission medium such as a wire, cable, or wireless transmission link. For instance, the various steps described throughout the present disclosure may be carried out by a single processor 175 (or computer subsystem) or, alternatively, multiple processors 175 (or multiple computer subsystems). Moreover, different sub-systems of the system 100 may include one or more computing or logic systems. Therefore, the above description should not be interpreted as a limitation on the present disclosure but merely an illustration.

The processor 175 may be in electronic communication with the first laser light source 121. For example, the processor 175 may be configured to send instructions to the first laser light source 121 to emit laser light, which produces the first laser beam 151 that is directed onto the first face 111 of the workpiece 110 by the first focusing lens 161.

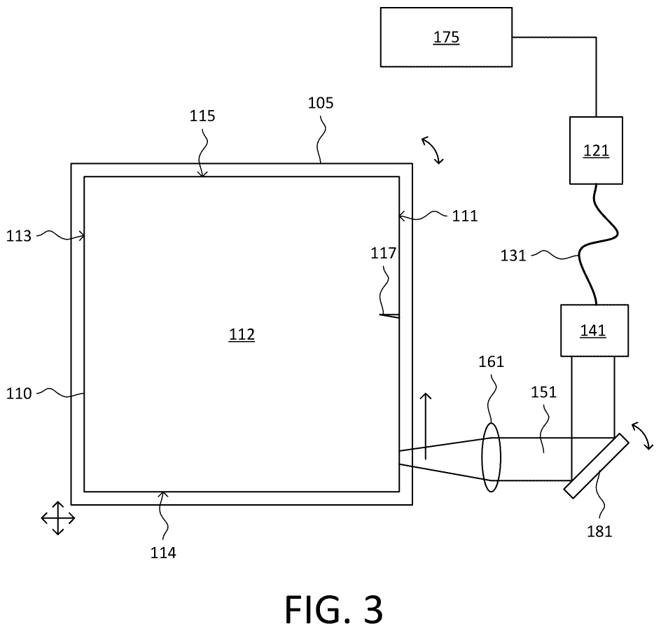

The processor 175 may be in electronic communication with the stage 105. For example, the processor 175 may be configured to send instructions to a motor or actuators of the stage 105 to cause the stage 105 to translate or rotate (e.g., in the directions shown in FIG. 1 and FIG. 3), which causes the first laser beam 151 to scan across the first face 111 of the workpiece 110.

In some embodiments, the system 100 may further comprise a first scanning mirror 181. The first scanning mirror 181 may be disposed between the first collimator 141 and the first focusing lens 161. The first scanning mirror 181 may be configured to move or rotate (e.g., as shown in FIG. 3) to change the direction of the first laser beam 151 passing through the first focusing lens 161. The processor 175 may be configured to send instructions to move the first scanning mirror 181. By moving the first scanning mirror 181, the laser spot from the first laser beam 151 may scan across the first face 111 of the workpiece 110.

The processor 175 may be in electronic communication with the thermal camera 170. For example, the processor 175 may be configured to send instructions to the thermal camera 170 to capture one or more thermal images 171 of the second face 112 of the workpiece 110. The thermal camera 170 may be configured to capture the one or more thermal images 171 of the second face 112 of the workpiece 110 synchronously with the first laser beam 151 being directed at the first face 111 of the workpiece 110. For example, the thermal camera 170 may capture the one or more thermal images 171 of the second face 112 of the workpiece 110 within a few tenths of a second after the first laser light source 121 is turned off. The specific timing of capturing the one or more thermal images 171 may depend on the properties of the first laser light source 121 and the thermal conductivity of the workpiece 110.

The processor 175 may be configured to determine presence of a crack 117 in the workpiece 110 based on the thermal image 171. The crack 117 may extend from the first face 111 into the workpiece 110 (e.g., across or beneath the second face 112 near the edge adjoining the first face 111 and the second face 112), as shown in FIG. 3. The processor 175 may be configured to obtain an intensity of each pixel of the thermal image along the edge of the workpiece 110 adjoining the first face 111 and the second face 112, which may include the edge itself and a perimeter surface of the second face 112 adjoining the edge (e.g., an area of 1 mm or less). The intensity of a pixel may correspond to a temperature of an area of the second face 112 of the workpiece 110, based on the heat generated by the laser spot from the first laser beam 151 on the first face 111. The processor 175 may be configured to derive a gradient map representing the spatial derivative of the temperature profile along the edge of the workpiece 110 adjoining the first face 111 and the second face 112 using the intensity of each pixel of the thermal image 171. For example, the intensity of each pixel along the edge of the workpiece 110 may be normalized based on the temperature distribution. A non-defective portion of the workpiece 110 may have a smooth transition in the temperature profile or the gradient map based on proximity to the laser spot. A crack 117 or defect in the workpiece 110 may spike or peak compared to the gradient of adjacent pixels. The processor 175 may be configured to determine that a crack 117 in the workpiece 110 exists where there is a peak in the gradient map. The processor 175 may use an edge detection algorithm or other algorithm to identify peaks in the gradient map. Accordingly, cracks 117 and other defects in the workpiece 110 can be identified.

The dimensions of the crack 117 that can be identified may depend on the resolution of the thermal camera 170. For example, a thermal camera 170 having 50 μm pixels may be used to detect a crack 117 having a thickness of less than 1 μm. In some embodiments, the crack 117 may be as thin as 100 nm may be detected using a thermal camera 170 having a pixel resolution of 5 to 200 μm.

While cracks 117 in the workpiece 110 are described, the system 100 can also be used to detect other types of defects in other types of workpieces. For example, the system 100 can be used for detection of air gaps in wafer-to-wafer and die-to-wafer bonding, detection of open circuits in metal lines of a PCB or FPD, detection of cracks in the different layers constituting solid state batteries or embedded semiconductor dies, delamination on PCBs, or other application use scenarios and is not limited herein.

In some embodiments, the first face 111 may have a smaller cross-sectional area than the second face 112. For example, as shown in FIG. 1, the first face 111 may correspond to a side wall of the workpiece 110, and the second face 112 may correspond to a top face of the workpiece 110. As shown in FIG. 3, the first focusing lens 161 may be configured to direct the first laser beam 151 onto the first face 111 of the workpiece 110 in a laser spot that has a diameter that is less than or equal to a thickness of the first face 111 of the workpiece 110. Accordingly, the laser spot from the first laser beam 151 may impinge on most of the thickness of the first face 111. By moving the stage 105 or the first scanning mirror 181, the first laser beam 151 can be scanned across the first face 111 of the workpiece 110 to generate heat along the thickness of the first face 111.

The first laser light source 121 may be operated in a continuous wave (CW) mode, combined with a continuous scanning of the first scanning mirror 181 or the stage 105, or it can be operated in a pulsed mode or a quasi-continuous wave (QCW) mode, with the first scanning mirror 181 or the stage 105 performing discrete steps between pulses, to illuminate discrete points on the workpiece 110.

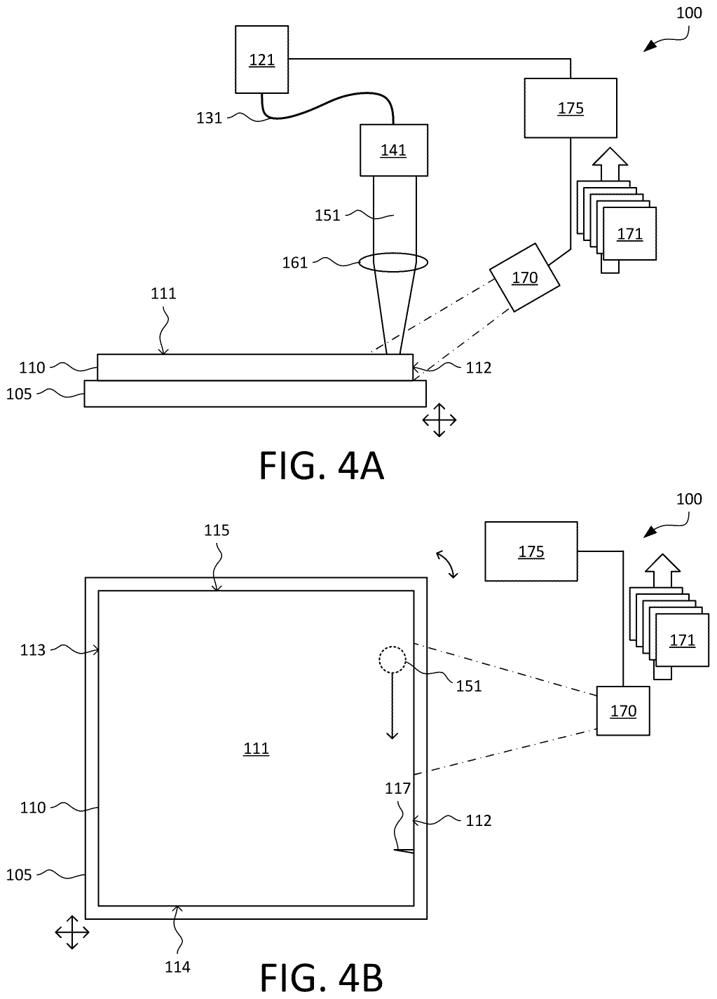

In some embodiments, the first face 111 may have a larger cross-sectional area than the second face 112. For example, as shown in FIG. 4A, the first face 111 may correspond to a top face of the workpiece 110, and the second face 112 may correspond to a side wall of the workpiece 110. Alternatively, the first face 111 may correspond to a bottom face of the workpiece 110. As shown in FIG. 4B, the first focusing lens 161 may be configured to direct the first laser beam 151 onto the first face 111 of the workpiece 110 near an edge adjoining the first face 111 and the second face 112. By moving the stage 105 or the first scanning mirror 181, the first laser beam 151 can be scanned across the first face 111 of the workpiece 110 to generate heat near the edge adjoining the first face 111 and the second face 112. In some embodiments, the thermal camera 170 may be directed at the first face 111 or the second face 112 to capture the one or more thermal images 171. Alternatively, the thermal camera 170 may be arranged at an angle in which at least a portion of both of the first face 111 and the second face 112 of the workpiece 110 are within the field of view of the thermal camera 170, such that the one or more thermal images 171 include the edge of the workpiece 110 adjoining the first face 111 and the second face 112.

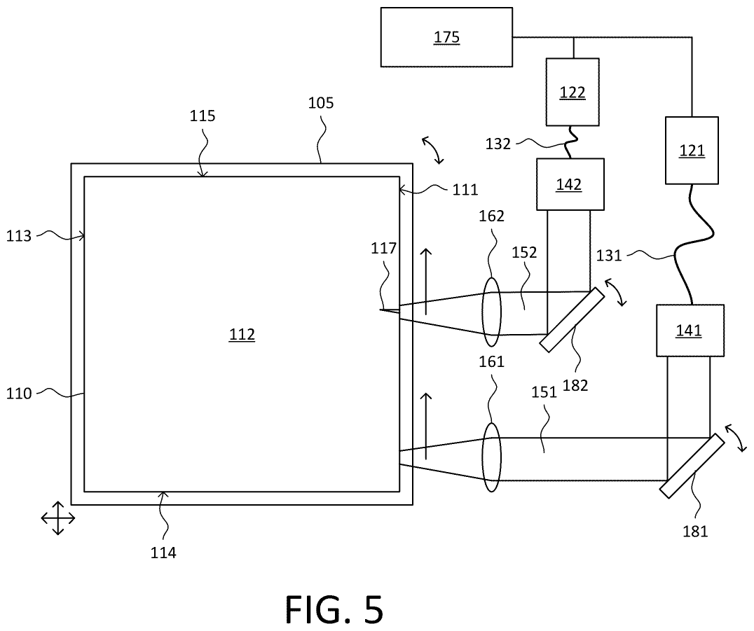

In some embodiments, the system 100 may further comprise additional laser light sources. For example, the system 100 may further comprise a second laser light source 122, as shown in FIG. 5. The second laser light source 122 may be configured to emit laser light and may be similar to the first laser light source 121. For example, the second laser light source 122 may be coupled to a second optical fiber 132, which may be configured to guide the laser light from the second laser light source 122 to a second collimator 142 to produce a second laser beam 152. Alternatively, the second laser beam 152 may be split from the first laser beam 151 using a beam splitter, a fiber splitter, a diffractive optical element, or other optical elements to use a single laser light source. A second focusing lens 162 may be configured to direct the second laser beam 152 onto the first face 111 of the workpiece 110 at a position that is offset from the second laser beam 152. For example, the first laser beam 151 may be directed at one end of the first face 111 and the second laser beam 152 may be directed at a midpoint of the first face 111. By moving the stage 105 or the first scanning mirror 181 and a corresponding second scanning mirror 182 (or a single scanning mirror), the first laser beam 151 and the second laser beam 152 can be scanned across the first face 111 of the workpiece 110. By using both the first laser light source 121 and the second laser light source 122, detection efficiency can be improved, as the scanning distance is decreased by the portions of the first face 111 impinged by laser spots from each laser light source. Additional laser light sources can be used illuminate the first face 111 of the workpiece with additional laser spots, which can further improve detection efficiency.

In some embodiments, after the system 100 detects cracks 117 on the first face 111 of the workpiece 110, the other sides of the workpiece 110 may be processed. For example, the processor 175 may be configured to send instructions to the stage 105 to rotate 90 degrees of 180 degrees (e.g., clockwise or counterclockwise from the position shown in FIG. 3 or FIG. 5), such that the first laser beam 151 is directed to each of the third face 113, fourth face 114, and fifth face 115 of the workpiece, and corresponding thermal images 171 are captured by the thermal camera 170 in each orientation to detect cracks 117 in each orientation of the workpiece 110.

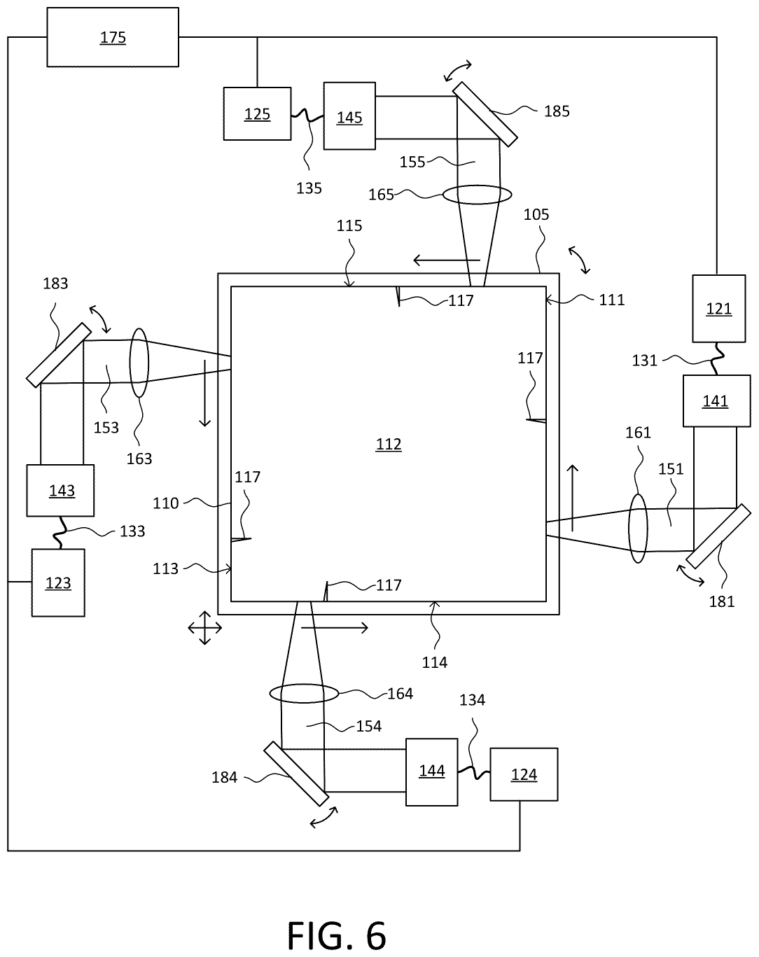

In some embodiments, the system 100 may be configured to simultaneously detect cracks 117 in multiple sides of the workpiece 110. For example, the system 100 may further comprise a third laser light source 123, a fourth laser light source 124, and a fifth laser light source 125, as shown in FIG. 6. The third laser light source 123, the fourth laser light source 124, and the fifth laser light source 125 may be configured to emit laser light similar to the first laser light source 121. For example, each of the third laser light source 123, the fourth laser light source 124, and the fifth laser light source 125 may be coupled to a third optical fiber 133, fourth optical fiber 134, and a fifth optical fiber 135, respectively, which may be configured to guide the laser light from each light source to a respective third collimator 143, fourth collimator 144, or fifth collimator 145 to produce a third laser beam 153, a fourth laser beam 154, and a fifth laser beam 155. Alternatively, the third laser beam 153, the fourth laser beam 154, and/or the fifth laser beam 155 may be split from the first laser beam 151 using a beam splitter, a fiber splitter, a diffractive optical element, or other optical elements to use fewer laser light sources. A third focusing lens 163 may be configured to direct the third laser beam 153 onto the third face 113 of the workpiece 110. The third face 113 may be orthogonal to the second face 112 and parallel to the first face 111. A fourth focusing lens 164 may be configured to direct the fourth laser beam 154 onto the fourth face 114 of the workpiece 110. The fourth face 114 may be orthogonal to the second face 112 and the first face 111. A fifth focusing lens 165 may be configured to direct the fifth laser beam 155 onto the fifth face 115 of the workpiece 110. The fifth face 115 may be orthogonal to the second face 112 and parallel to the fourth face 114. A third scanning mirror 183, a fourth scanning mirror 184, and a fifth scanning mirror 185 can be used to scan the laser spots from the third laser beam 153, the fourth laser beam 154, and the fifth laser beam 155 across respective ones of the third face 113, the fourth face 114 and the fifth face 115 of the workpiece 110. By using the first laser light source 121, the third laser light source 123, the fourth laser light source 124, and the fifth laser light source 125, detection efficiency can be improved, as the stage 105 can remain stationary while each of the first face 111, the third face 113, the fourth face 114, and fifth face 115 of the workpiece 110 are impinged by laser spots from each laser light source and a single thermal image 171 can be processed to detect defects along each face.

In some embodiments, the system 100 may include the first laser light source 121 and the third laser light source 123, which impinge the first laser beam 151 and the third laser beam 153 on the parallel first face 111 and third face 113 of the workpiece. Then, by rotating the stage 105 by 90 degrees, the first laser beam 151 and the third laser beam 153 may impinge on the parallel fourth face 114 and fifth face 115, which can improve detection efficiency without the need for the fourth laser light source 124 and the fifth laser light source 125.

In some embodiments, the system 100 may include a second laser light source 122 can be included in parallel to each of the third laser light source 123, the fourth laser light source 124, and the fifth laser light source 125 to illuminate each of the third face 113, the fourth face 114, and the fifth face 115 of the workpiece 110 with additional laser spots, which can further improve detection efficiency.

With the system 100, the first laser light source 121 can generate heat along a first face 111 of a workpiece 110 with a first laser beam 151 while a thermal camera 170 captures a thermal image 171 of a second face 112 of the workpiece 110, and the thermal image 171 can be used to detect cracks 117 and other defects in the workpiece 110 at the interface between the first face 111 and the second face 112. The system 100 can be applied to opaque workpieces 110 with rapid scans for rapid inspection.

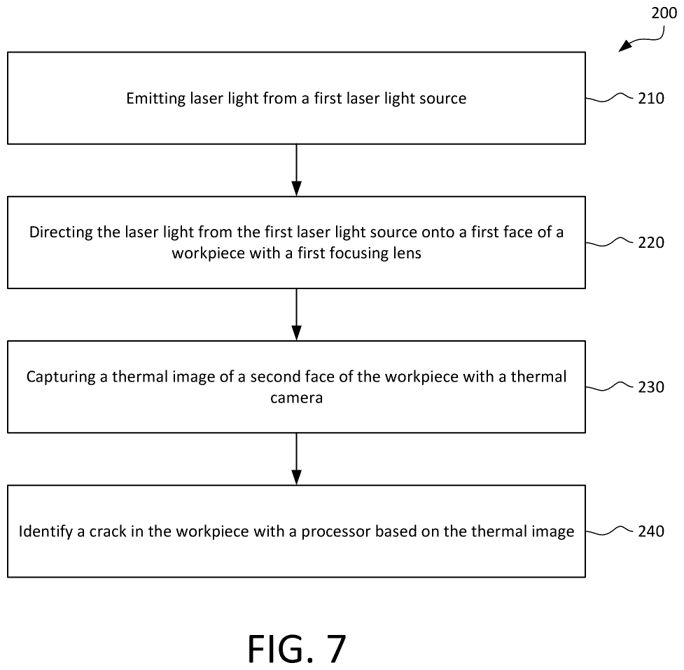

Another embodiment of the present disclosure provides a method 200. As shown in FIG. 7, the method 200 may comprise the following steps.

At step 210, laser light is emitted from a first laser light source. The first laser light source may be a laser diode or other laser light source configured to emit laser light. The laser light may be a pulsed laser or a continuous laser beam. The first laser light source may be coupled to a first optical fiber. The first optical fiber may guide the laser light emitted by the first laser light source to a first collimator to produce a first laser beam.

At step 220, the laser light from the first laser light source is directed onto a first face of a workpiece with a first focusing lens. For example, the first focusing lens may direct a laser spot from the first laser beam onto the first face of the workpiece. In some embodiments, the diameter of the laser spot may be less than or equal to a thickness of the first face. For example, the diameter of the laser spot may be 25 to 750 μm. By directing the first laser beam onto the first face of the workpiece, heat may be generated in a non-uniform temperature profile from the laser spot into the workpiece.

At step 230, a thermal image of a second face of the workpiece is captured with a thermal camera. The thermal camera may be a forward looking infrared (FLIR) camera or other type of non-contact temperature measurement sensor or an array of sensors. The thermal camera may be directed toward the second face of the workpiece. The second face may be orthogonal to the first face. In other words, the first face and the second face may be adjacent faces of the workpiece joined at an edge. The heat generated by the laser spot from the first laser beam on the first face may have a non-uniform temperature profile that extends into the workpiece. Thus, the thermal image may reflect the non-uniform temperature profile on the second face of the workpiece (e.g., with the highest temperature at the edge of the second face and the temperature being reduced farther away from the edge adjoining the first face).

At step 240, a processor identifies a crack in the workpiece based on the thermal image. The crack may extend from the first face into the workpiece (e.g., across or beneath the second face near the edge adjoining the first face and the second face).

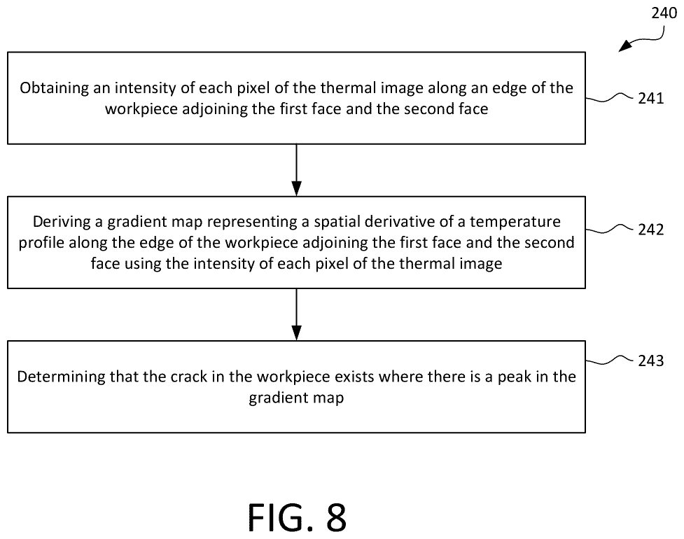

In some embodiments, step 240 may comprise the following steps, as shown in FIG. 8.

At step 241, an intensity is obtained of each pixel of the thermal image along an edge of the workpiece adjoining the first face and the second face. The intensity of a pixel may correspond to a temperature of an area of the second face of the workpiece, based on the heat generated by the laser spot from the first laser beam on the first face.

At step 242, a gradient map representing a spatial derivative of a temperature profile along the edge of the workpiece adjoining the first face and the second face is derived using the intensity of each pixel of the thermal image. A non-defective portion of the workpiece may have a smooth transition in the temperature profile based on proximity to the laser spot. A crack or defect in the workpiece may have a spike or peak in the intensity compared to the gradient of adjacent pixels.

At step 243, the crack in the workpiece is determined to exist where there is a peak in the gradient map. For example, an edge detection algorithm or other type of algorithm can be used to identify peaks in the gradient map. Accordingly, cracks and other defects in the workpiece can be identified. In some embodiments, the crack may be of submicron thickness.

While cracks in the workpiece are described, the method 200 can also be used to detect other types of defects in other types of workpieces. For example, the method 200 can be used for detection of air gaps in wafer-to-wafer and die-to-wafer bonding, detection of open circuits in metal lines of a PCB or FPD, detection of cracks in different layers constituting solid state batteries or embedded semiconductor dies, delamination on PCBs, or other application use scenarios and is not limited herein.

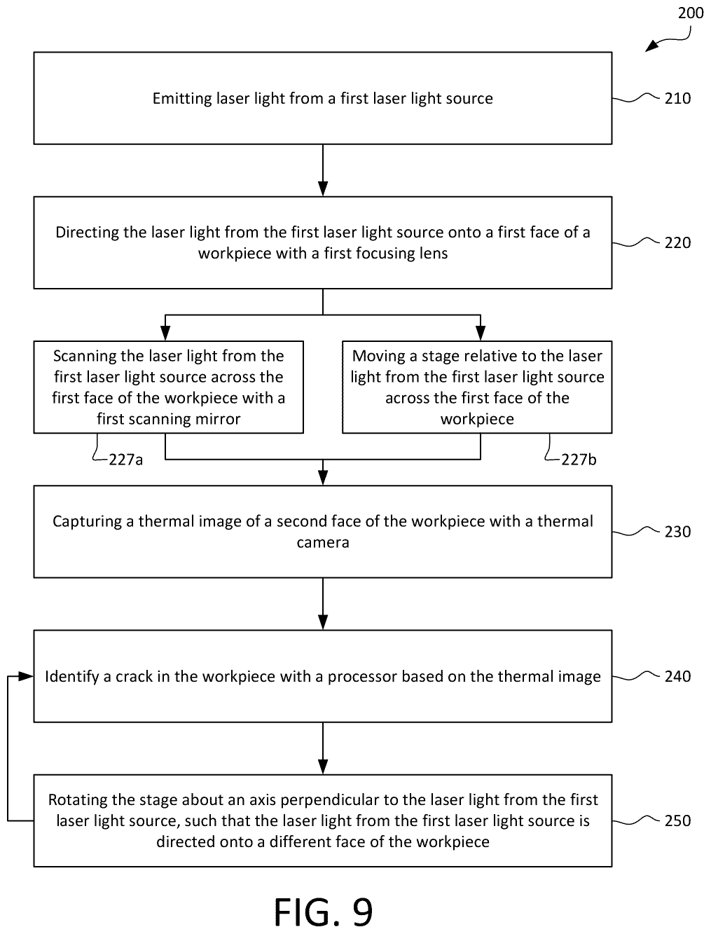

In some embodiments, the method 200 may further comprise step 227a or step 227b, as shown in FIG. 9.

At step 227a, the laser light from the first laser light source is scanned across the first face of the workpiece with a first scanning mirror. The first scanning mirror may be disposed between the first collimator and the first focusing lens. The first scanning mirror may be configured to move or rotate to change the direction of the first laser beam passing through the first focusing lens. By moving the first scanning mirror, the laser spot from the first laser beam may scan across the first face of the workpiece.

At step 227b, a stage is moved relative to the laser light to scan the laser light from the first laser light source across the first face of the workpiece. For example, motor or actuators of the stage can cause the stage to translate or rotate, which causes the first laser beam to scan across the first face of the workpiece. In some embodiments where the workpiece is cylindrical, rotating the stage relative to the laser light can scan the laser light across an annular face of the workpiece.

In some embodiments, the method 200 may further comprise step 250 shown in FIG. 9. At step 250, the stage is rotated about an axis perpendicular to the laser light from the first laser light source, such that the laser light from the first laser light source is directed onto a different face of the workpiece. For example, by rotating the stage 90 degrees, the laser light from the first laser light source may be directed onto the fourth face or the fifth face of the workpiece, and by rotating the stage 180 degrees, the laser light from the first laser light source may be directed onto the third face of the workpiece. Thus, with consecutive 90-degree rotations, the laser light from the first laser light source can be scanned across the first face, the third face, the fourth face, and the fifth face of the workpiece. Steps 230 may be repeated after each rotation to capture a thermal image of the corresponding face of the workpiece and identify cracks based on the images.

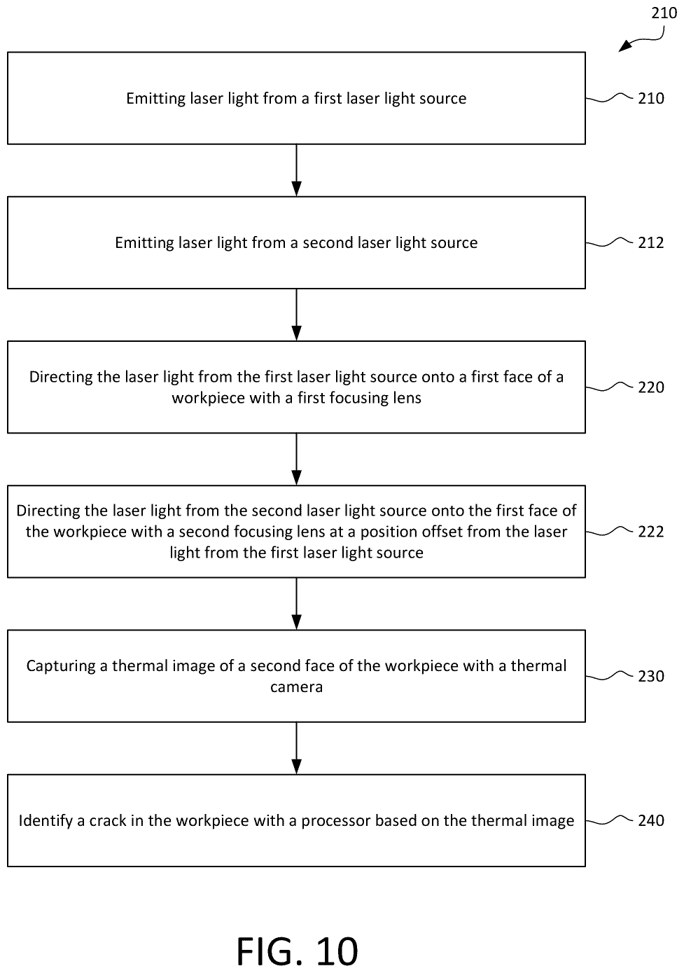

In some embodiments, the method 200 may further comprise step 212 and step 222, as shown in FIG. 10.

At step 212, laser light is emitted from a second laser light source. The second laser light source may be configured to emit laser light and may be similar to the first laser light source. For example, the second laser light source may be coupled to a second optical fiber, which may be configured to guide the laser light from the second laser light source to a second collimator to produce a second laser beam. In some embodiments, the second laser beam may be produced by splitting the first laser beam with a beam splitter, a fiber splitter, a diffractive optical element, or other optical element instead of using a second laser source.

At step 222, the laser light from the second laser light source is directed onto the first face of the workpiece with a second focusing lens at a position that is offset from the laser light from the first laser light source. For example, the first laser beam may be directed at one end of the first face and the second laser beam may be directed at a midpoint of the first face.

By moving the stage or the first scanning mirror and a corresponding second scanning mirror (or a single scanning mirror), the first laser beam and the second laser beam can be scanned across the first face of the workpiece. By using both the first laser light source and the second laser light source, detection efficiency can be improved, as the scanning distance is decreased by the portions of the first face impinged by laser spots from each laser light source. Additional laser light sources can be used illuminate the first face of the workpiece with additional laser spots, which can further improve detection efficiency.

Although FIG. 10 illustrates the steps 210 and 212 as being performed simultaneously, these steps may be performed at different times and is not limited herein. Similarly, the sequence of steps 220 and 222 may depend on the sequence of preceding steps 210 and 212 and is not limited herein.

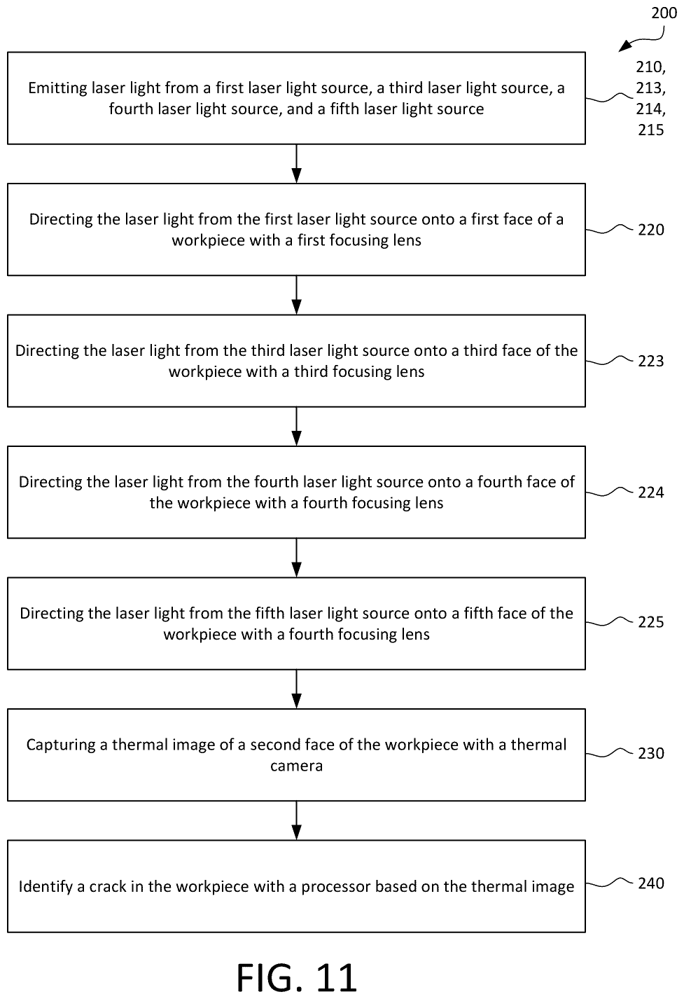

In some embodiments, the method 200 may further comprise step 213 and step 223, as shown in FIG. 11.

At step 213, laser light is emitted from a third laser light source. The third laser light source may be configured to emit laser light and may be similar to the first laser light source. For example, the third laser light source may be coupled to a third optical fiber, which may be configured to guide the laser light from the third laser light source to a third collimator to produce a third laser beam.

At step 223, the laser light from the third laser light source is directed onto a third face of the workpiece with a third focusing lens. For example, the third focusing lens may direct the third laser beam onto the third face of the workpiece. The third face may be orthogonal to the second face and parallel to the first face.

A third scanning mirror can be used to scan the laser spot from the third laser beam across the third face of the workpiece. Accordingly, detection efficiency can be improved, as both the first face and the third face can be impinged with laser spots from each laser light source simultaneously. The stage can then be rotated to further impinge the adjacent fourth face and fifth face of the workpiece.

In some embodiments, the method 200 may further comprise the following steps.

At step 214, laser light is emitted from a fourth laser light source. The fourth laser light source may be configured to emit laser light and may be similar to the first laser light source. For example, the fourth laser light source may be coupled to a fourth optical fiber, which may be configured to guide the laser light from the fourth laser light source to a fourth collimator to produce a fourth laser beam.

At step 215, laser light is emitted from a fifth laser light source. The fifth laser light source may be configured to emit laser light and may be similar to the first laser light source. For example, the fifth laser light source may be coupled to a fifth optical fiber, which may be configured to guide the laser light from the fifth laser light source to a fifth collimator to produce a fifth laser beam.

A step 224, the laser light from the fourth laser light source is directed onto a fourth face of the workpiece with a fourth focusing lens. For example, the fourth focusing lens may direct the fourth laser beam onto the fourth face of the workpiece. The fourth face may be orthogonal to the second face and the first face.

At step 225, the laser light from the fifth laser light source is directed onto a fifth face of the workpiece with a fifth focusing lens. For example, the fifth focusing lens may direct the fifth laser beam onto the fifth face of the workpiece. The fifth face may be orthogonal to the second face and parallel to the fourth face.

A fourth scanning mirror and a fifth scanning mirror can be used to scan the laser spots from the fourth laser beam and the fifth laser beam across the fourth face and the fifth face of the workpiece. Accordingly, detection efficiency can be improved, as each of the first face, the third face, the fourth face, and the fifth face can be impinged with laser spots from each laser light source simultaneously while the stage remains stationary, and a single thermal image can be processed to detect defects along each face.

Although FIG. 11 illustrates the steps 210, 213, 214, and 215 as being performed simultaneously, these steps may be performed at different times and is not limited herein. Similarly, the sequence of steps 220, 223, 224, and 225 may depend on the sequence of preceding steps 210, 213, 214, and 215 and is not limited herein.

With the method 200, a laser light source can generate heat along a first face of a workpiece while a thermal camera captures a thermal image of a second face of the workpiece, and the thermal image can be used to detect cracks and other defects in the workpiece at the interface between the first face and the second face. The method 200 can be applied to opaque workpieces with rapid scans for rapid inspection.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

What is claimed is:

1. A system comprising:
a first laser light source configured to emit laser light;
a first focusing lens configured to direct the laser light from the first laser light source onto a first face of a workpiece;
a thermal camera configured to capture a thermal image of a second face of the workpiece, wherein the second face is orthogonal to the first face; and
a processor configured to identify a crack in the workpiece based on the thermal image.

2. The system of claim 1, further comprising:
a first scanning mirror configured to direct the laser light from the first laser light source to scan across the first face of the workpiece.

3. The system of claim 1, further comprising:
a stage, wherein the workpiece is disposed on the stage and the stage is movable to scan the laser light from the first laser light source across the first face of the workpiece.

4. The system of claim 1, wherein the first face has a smaller cross-sectional area than the second face.

5. The system of claim 4, wherein the laser light has a diameter that is less than or equal to a thickness of the first face of the workpiece.

6. The system of claim 1, wherein the first face has a larger cross-sectional area than the second face.

7. The system of claim 6, wherein the first focusing lens is configured to direct the laser light onto the first face of the workpiece near an edge adjoining the first face and the second face.

8. The system of claim 1, further comprising:
a second laser light source configured to emit laser light; and
a second focusing lens configured to direct the laser light from the second laser light source onto the first face of the workpiece at a position that is offset from the laser light from the first laser light source.

9. The system of claim 1, further comprising:
a third laser light source configured to emit laser light; and
a third focusing lens configured to direct the laser light from the third laser light source onto a third face of the workpiece, wherein the third face is orthogonal to the second face and parallel to the first face.

10. The system of claim 9, further comprising:
a fourth laser light source configured to emit laser light;
a fourth focusing lens configured to direct the laser light from the fourth laser light source onto a fourth face of the workpiece, wherein the fourth face is orthogonal to the second face and the first face;
a fifth laser light source configured to emit laser light; and
a fifth focusing lens configured to direct the laser light from the fifth laser light source onto a fifth face of the workpiece, wherein the fifth face is orthogonal to the second face and parallel to the fourth face.

11. The system of claim 1, wherein the thermal camera is a forward looking infrared (FLIR) camera.

12. The system of claim 1, wherein the processor is configured to identify the crack in the workpiece based on the thermal image by:
obtaining an intensity of each pixel of the thermal image along an edge of the workpiece adjoining the first face and the second face;
deriving a gradient map representing a spatial derivative of a temperature profile along the edge of the workpiece adjoining the first face and the second face using the intensity of each pixel of the thermal image; and
determining that the crack in the workpiece exists where there is a peak in the gradient map.

13. A method comprising:
emitting laser light from a first laser light source;
directing the laser light from the first laser light source onto a first face of a workpiece with a first focusing lens;
capturing a thermal image of a second face of the workpiece with a thermal camera, wherein the second face is orthogonal to the first face; and
identifying, with a processor, a crack in the workpiece based on the thermal image.

14. The method of claim 13, further comprising:
scanning the laser light from the first laser light source across the first face of the workpiece with a first scanning mirror.

15. The method of claim 13, further comprising:
moving a stage relative to the laser light to scan the laser light from the first laser light source across the first face of the workpiece, wherein the workpiece is disposed on the stage.

16. The method of claim 13, further comprising:
emitting laser light from a second laser light source; and
directing the laser light from the second laser light source onto the first face of the workpiece with a second focusing lens at a position that is offset from the laser light from the first laser light source.

17. The method of claim 13, further comprising:
emitting laser light from a third laser light source; and
directing the laser light from the third laser light source onto a third face of the workpiece with a third focusing lens, wherein the third face is orthogonal to the second face and parallel to the first face.

18. The method of claim 17, further comprising:
emitting laser light from a fourth laser light source;
directing the laser light from the fourth laser light source onto a fourth face of the workpiece with a fourth focusing lens, wherein the fourth face is orthogonal to the second face and the first face;
emitting laser light from a fifth laser light source; and
directing the laser light from the fifth laser light source onto a fifth face of the workpiece with a fifth focusing lens, wherein the fifth face is orthogonal to the second face and parallel to the fourth face.

19. The method of claim 13, wherein the workpiece is disposed on a stage, and the method further comprises:
rotating the stage about an axis perpendicular to the laser light from the first laser light source, such that the laser light from the first laser light source is directed onto a fourth face of the workpiece with the first focusing lens, wherein the fourth face is orthogonal to the second face and the first face.

20. The method of claim 13, wherein identifying, with the processor, the crack in the workpiece based on the thermal image comprises:
obtaining an intensity of each pixel of the thermal image along an edge of the workpiece adjoining the first face and the second face;
deriving a gradient map representing a spatial derivative of a temperature profile along the edge of the workpiece adjoining the first face and the second face using the intensity of each pixel of the thermal image; and
determining that the crack in the workpiece exists where there is a peak in the gradient map.
